(19) United States
(12) Patent Application Publication
(10) Pub. No.: US 2024/0024589 A1
(43) Pub. Date: Jan. 25, 2024

(54) STERILE DRUG CHAMBER VALVE

(71) Applicant: West Pharmaceutical Services, Inc., Exton, PA (US)

(72) Inventors: Ran HEZKIAHU, Scottsdale, AZ (US); Tommy Gene DAVIS, Athens, TX (US)

(21) Appl. No.: 18/355,487

(22) Filed: Jul. 20, 2023

Related U.S. Application Data
(60) Provisional application No. 63/369,122, filed on Jul. 22, 2022.

(51) Int. Cl. A61M 5/32

(52) U.S. Cl. CPC A61M 5/3293; A61M 2005/3128

(57) ABSTRACT

There are provided devices and methods for the selective opening and closing of a medicament container using a valve structure. The valve structure may include a seal which is rotatable to selectively align an opening with a fluid path in a valve structure. By rotating the seal, a user may open a medicament container at the point of use, thereby maintaining sterility of one or more components of a medicament delivery device.

### CROSS REFERENCE TO RELATED APPLICATIONS

This application claims the benefit of U.S. Provisional Patent App. No. 63/369,122, filed Jul. 22, 2022, the disclosure of which is hereby incorporated by reference herein.

### TECHNICAL FIELD

The present disclosure relates to a valve structure for selectively connecting a medicament container to a delivery conduit, or to a device for delivering medicament, and to a method for manufacturing a valve structure for selectively connecting a medicament container to a delivery conduit. More particularly, this disclosure relates to a rotatable valve structure that can be moved between an opened and closed position. The structures and methods disclosed herein may be implemented in an injection device, for example an auto-injector or an infusion device.

### BACKGROUND

One of the challenges posed by many injection devices is that component parts are manufactured and sterilized separately. Typically, the containers are sterilized (e.g., with electron-beam processing) and then sent for filling with medicament. The filled container is then sealed with a septum and packed separately from the injection device (comprising a delivery conduit), which is sterilized with gas. The user must first sterilize the septum before inserting it into the injection device for use. This increases the complexity of use and may lead to higher chances of contamination, especially for minimally trained health workers or patients who self-administer their medicaments.

Pre-filled syringes reduce the complexity of the steps that need to be taken at the point of care. However, this solution cannot be employed in all drug delivery contexts. For example, products with long fluid path tubing (e.g., infusion devices in which the needle is remote from the medicament container) may be hard to handle during supply chain and assembly. Moreover, in such devices, the medicament container and the needle may not be brought into fluid communication until the device is ready to be used.

A need therefore exists for an improved connection between the medicament container and delivery conduit.

### SUMMARY

In a first aspect of the disclosure, there is provided a valve structure for selectively connecting a medicament container to a delivery conduit, the valve structure comprising: a valve housing comprising a first portion that is configured to couple to a medicament container, a second portion that is configured to couple to a delivery conduit, and a first opening providing a fluid pathway from the first portion to the second portion; and a seal extending across the first opening for selectively sealing the first opening in the valve housing, the seal comprising a second opening extending therethrough,
wherein the seal is rotatable relative to at least one of the first portion and the second portion of the valve housing between a first position in which the second opening is not in fluid communication with the first opening and a second position in which the seal is rotated such that the second opening is in fluid communication with the first opening. In this way, the valve structure may allow a user to selectively open or close a medicament container connected to the valve structure. Selective opening and/or closing of a medicament container may allow a user to optionally use a portion of medicament in a container whilst maintaining sterility of the medicament container. Selective opening and/or closing of the medicament container using the valve structure may provide for users with reduced dexterity the ability to maintain the sterility and closure of the medicament container until the point of use.

Optionally, the medicament container may be opened by actuating the valve structure. A user may operate the valve structure to cause the medicament container to open. The actuation may include operating a button, lever, or the like.

In a further aspect, the at least one of the first portion and the second portion of the valve housing may further comprise a sealing seat surrounding the first opening in the valve housing, against which the seal is seated. The second portion may be coupled to a delivery conduit via a delivery conduit hub, wherein the second opening in the seal is in fluid communication with the delivery conduit.

The first opening may have a first central longitudinal axis, the seal having a second central longitudinal axis, the second opening having a third central longitudinal axis, and the second central longitudinal axis being offset from the first and third central longitudinal axes in a transverse direction, or non-coaxial with the first and third central longitudinal axes.

In a further aspect, the seal may be fixedly coupled to the second portion of the valve housing. The seal and the second portion of the valve housing may be rotatable together relative to the first portion of the valve housing. In this way, the second portion of the valve housing may be used in order to effect rotation of the seal. Further, the seal and the second portion of the valve housing may be keyed such that they are rotatable together.

In a further aspect, the second opening may comprise a central channel and a connecting channel that is in fluid communication with the central channel. Optionally, the second opening is arranged such that when the seal is in the second position, the connecting channel is in fluid communication with the first opening and there is fluid communication between the first opening and the central channel, and when the seal is in the first position, the connecting channel is not in fluid communication with the first opening and there is no fluid communication between the first opening and the central channel. Further optionally, the seal comprises a body having a first face, and the central channel extends from the first face and at least partially through the body of the seal. The body of the seal may have a second face, and the connecting channel may comprise a slot extending from the second face to the central channel. The slot may extend radially from a central axis of the seal to an eccentric position. In this way, the seal may provide a fluid path which maintains the delivery conduit centrally with the medicament container. This may be advantageous, for example, where a needle is located on the end of the medicament container and a user benefits from the needle being aligned with the medicament container.

Optionally, the first portion comprises a flange, the second portion comprises a corresponding detent, and the flange and the detent are configured to rotatably couple the first portion and the second portion.

Further optionally, the valve structure comprises a stop assembly configured to limit rotation of the seal between the first position and the second position. In this way, a user of the valve structure may be provided with physical feedback when either of the first and second positions are reached. The stop assembly may also prevent a user from over-rotating the valve structure beyond the desired first or second positions. Optionally, the stop assembly comprises a pin fixed to one of the first portion and the second portion, the pin being received in a corresponding slot of the other of the first portion and the second portion, such that the slot restricts rotation of the second portion beyond the first position or the second position.

In a further aspect, the first central longitudinal axis, the second central longitudinal axis and the third central longitudinal axis may be parallel to each other.

Optionally, the delivery conduit hub is a needle hub comprising a needle. Further optionally, the needle is aligned with the second central longitudinal axis of the seal. Optionally, the seal is a circular disc.

In a further aspect, the first portion comprises a first generally cylindrical body and/or the second portion comprises a second generally cylindrical body. Optionally, the first generally cylindrical body comprises a fourth central longitudinal axis and is substantially co-axial with the first central longitudinal axis.

In a further aspect, the sealing seat provides a sealing surface disposed in the second portion of the valve housing. Optionally, the second opening is in registry with the sealing surface when the seal is in the first position.

In a further aspect, the first portion comprises a connector for coupling the valve structure to a medicament container.

Optionally, the seal comprises an elastomeric material.

In another aspect, there is provided a device for delivering medicament comprising: the valve structure according to any of the aspects above and a medicament container comprising a third opening coupled to the first portion and in fluid communication with the first opening in the valve housing. Optionally, the third opening is surrounded by a container sealing surface and the seal is configured to seal against the container sealing surface. Further optionally, one of the medicament container and the second portion of the valve housing comprises a male luer connecting portion and the other of the medicament container and the second portion of the valve housing comprises a female luer connecting portion. Further optionally, the engagement between the medicament container and the second portion of the valve housing can comprise a snap fit, a friction fit, an adhesive, an over-molded connected, or the like. Optionally, the engagement between the medicament container and the valve housing may be designed to be permanent and irreversible so that the medicament container cannot be readily separated from the valve housing.

In a further aspect, the device may comprise an injection needle in fluid communication with the delivery conduit hub. Optionally, the injection needle is remote from the delivery conduit hub and is connected thereto via a flexible delivery conduit.

In a further aspect, the device may comprise a removable cap. Optionally, the removable cap is configured to rotate the seal from the first position to the second position. Further optionally, the cap is removable by rotation and is configured to rotate the seal between the first and second position.

In a further aspect, the removable cap may comprise a first engagement member; the valve structure may comprise a second engagement member, and the first engagement member may be configured to engage the second engagement member during removal of the cap to move the seal from the first position to the second position.

In a further aspect, there is provided a method for manufacturing a valve structure for selectively connecting a medicament container to a delivery conduit, the method comprising: providing a valve housing comprising a first portion that is configured to couple to a medicament container, a second portion that is configured to couple to a delivery conduit, and a first opening providing fluid pathway from the first portion to the second portion; and providing a seal extending across the first opening for selectively sealing the first opening in the valve housing, the seal comprising a second opening extending therethrough, wherein the seal is rotatable relative to at least one portion of the first portion and the second portion of the valve housing between a first position in which the seal is oriented such that the second opening is not in fluid communication with the first opening and a second position in which the seal is rotated such that the second opening is in fluid communication with the first opening.

Optionally, the method further comprises providing a medicament container comprising a third opening surrounded by a container sealing surface, wherein the seal is configured to seal against the container sealing surface. Further optionally, the method further comprises providing a delivery conduit hub, fixedly connected to the seal, and in fluid communication with the second opening.

Further optionally, the method further comprises the step of providing a flexible delivery conduit coupled to the delivery conduit hub and further comprising providing a needle in fluid communication with the flexible delivery conduit, wherein the step of providing the needle in fluid communication with the flexible delivery conduit optionally includes inserting a proximal end of the needle into the flexible delivery conduit and overmolding a collar over a portion of the needle and the flexible delivery conduit to form a seal.

In a further aspect there is provided a method of operating a valve structure according to any of the aspects described above, comprising:
rotating the seal relative to at least one of the first portion and the second portion of the valve housing from the first position in which the second opening is not in fluid communication with the first opening and to the second position in which the seal is rotated such that the second opening is in fluid communication with the first opening.

In a further aspect, the method of operating the valve structure optionally comprises ejecting the medicament from the medicament container. Optionally, the medicament may be delivered to one of a human or animal body, or to an injection training model.

In a further aspect, the method of operating the valve structure optionally comprises rotating the seal relative to at least one of the first portion and the second portion of the valve housing from the second position in which the seal is rotated such that the second opening is in fluid communication with the first opening and to the first position in which the second opening is not in fluid communication with the first opening.

### BRIEF DESCRIPTION OF THE DRAWINGS

Aspects of the disclosure will now be described in more detail with reference to a number of non-limiting, exemplary embodiments shown in the following drawings, in which:

FIG. 1 shows an exploded perspective view of an injection assembly;

FIG. 2a shows an exploded perspective view of the valve structure of FIG. 1;

FIG. 2b shows a cross-section side-view of the valve structure of FIG. 1;

FIG. 3 shows a cross-section perspective view of the injection assembly of FIG. 1;

FIG. 4a shows a cross-section perspective view of the medicament container and the valve structure, the seal in the first position;

FIG. 4b shows a cross-section perspective view of the medicament container and the valve structure, the seal in the second position;

FIG. 5a shows a cross-section perspective view of the injection assembly, the seal in the first position;

FIG. 5b shows a cross-section perspective view of the injection assembly, the seal in the second position;

FIG. 6 shows a perspective view of the injection assembly and the removable cap;

FIG. 7a shows an exploded perspective view of an embodiment of the valve structure;

FIG. 7b shows a cross-section side-view of the valve structure of FIG. 7a;

FIG. 8a shows a perspective view of the second portion and the seal;

FIG. 8b shows an exploded perspective cross-section view of the second portion and the seal of FIG. 8a;

FIG. 9 shows a perspective view of the valve structure of FIG. 7a;

FIG. 10 shows an injection assembly comprising a flexible medicament container;

FIG. 11a shows an injection assembly comprising an injection needle that is remote from a needle, where the flexible delivery conduit is extended;

FIG. 11b shows an injection assembly comprising an injection needle that is remote from a needle, where the flexible delivery conduit is retracted;

FIGS. 12a-12c illustrate different configurations of an injector body that is connected to an infusion set body; and

FIG. 13 is a flowchart illustrating a method for manufacturing a valve structure for selectively connecting a medicament container to a delivery conduit according to the present disclosure.

Like reference numerals are used for like components throughout the drawings.

### DETAILED DESCRIPTION

The present disclosure is directed generally to an injection device that includes a rotatable valve structure which is configured to selectively connect a medicament container to a delivery conduit via means of a seal that is rotatable between an open position and closed position. An associated method is also provided.

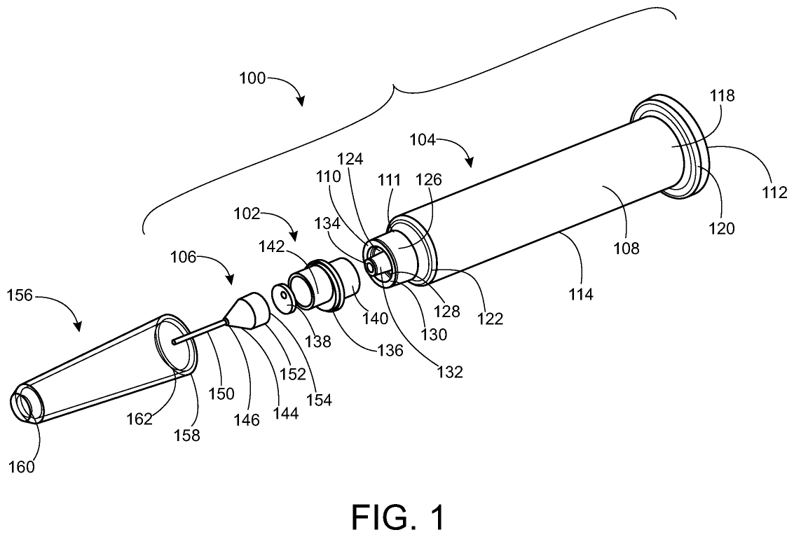

FIG. 1 shows an exploded perspective view of an embodiment of an injection assembly 100. The injection assembly 100 comprises a valve structure 102; a medicament container 104 and a delivery conduit hub 106. Optionally, the injection assembly 100 also comprises a removable cap 156.

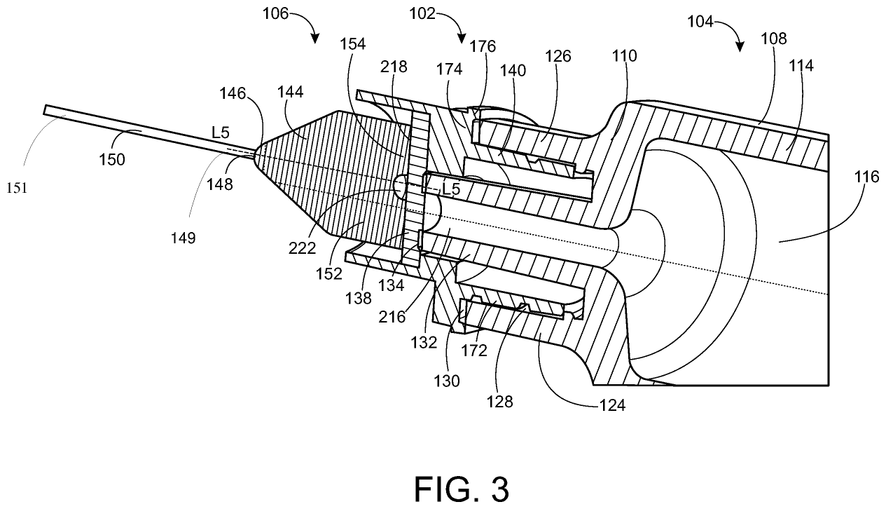

As shown in FIG. 1, the medicament container 104 comprises a barrel 108 extending from a proximal end 112 of the medicament container 104 towards a distal end 110 of the medicament container 104. The barrel 108 includes a cylindrical wall 114 which defines an interior volume 116 (shown in FIG. 3). The interior volume 116 is configured to contain one or more liquid medicaments. The barrel 108 is open at the proximal end 112, such that a plunger (not shown) can be inserted to seal the barrel 108 at the proximal end 112 and optionally exert pressure on a medicament contained in the interior volume 116 to eject the medicament from an opening 216 (shown in FIG. 3) at the distal end 110 of the medicament container 104.

At a distal end 111 of the barrel 108, the medicament container 104 comprises a male Luer fitting 124. The male Luer fitting 124 comprises a sleeve portion 126. The sleeve portion 126 may be formed as a hollow cylinder that extends from the distal end 111 of the barrel 108 to an outer sealing surface 130 at the distal end 110 of the medicament container 104. The sleeve portion 126 comprises a Luer thread 128 disposed on its inner surface. The male Luer fitting 124 also comprises a conduit portion 132. The conduit portion 132 may be formed as a hollow cylinder that extends from the distal end 111 of the barrel 108 and terminates at a container sealing surface 134. The conduit portion 132 is positioned within the sleeve portion 126 such that the conduit portion 132 is surrounded by the sleeve portion 126. The male Luer fitting 124 may be co-molded with the barrel 108 of the medicament container 104, or the male Luer fitting 124 may be formed separately and joined to the barrel 108.

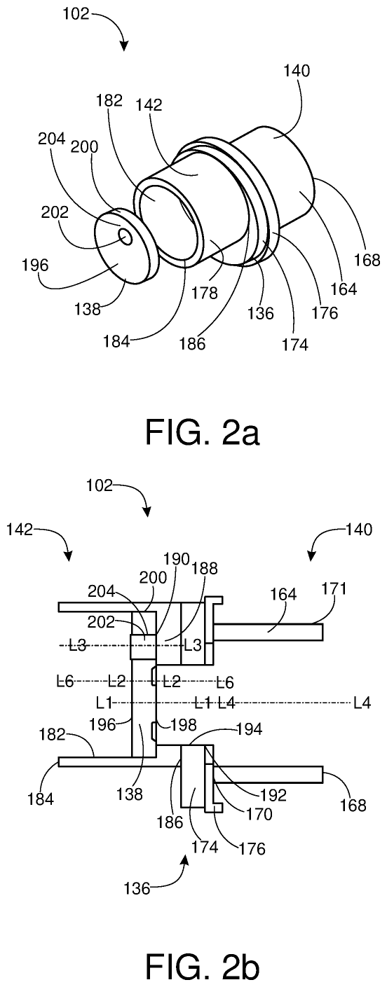

The valve structure 102 comprises a valve housing 136 and a seal 138. The valve housing 136 comprises a first portion 140 and a second portion 142. The first portion 140 is configured to connect to the distal end 110 of the medicament container 104 via the Luer fitting 124. The connection between the first portion 140 and the medicament container 104 is further described with reference to FIG. 3. The first portion 140 is connected to the second portion 142. The second portion 142 houses the seal 138. The valve structure 102 will be discussed in more detail with reference to FIG. 2a and FIG. 2b.

It should be appreciated that in some aspects, the engagement between the medicament container and the valve housing can comprise a snap fit, a friction fit, an adhesive, an over-molded connected, or the like.

The delivery conduit hub 106 comprises a conical portion 144 that tapers to a distal end 146. The distal end 146 of the conical portion 144 comprises an opening 148 (shown in FIG. 3). A delivery conduit 150 may be embedded in the opening. The delivery conduit 150 shown in FIG. 1 is a needle, but any suitable delivery conduit may be used, such as a tube-needle combination. The delivery conduit 150 has a proximal end 149 which is embedded in the opening 148 and extends to a distal end 151 of the delivery conduit 150. The base of the conical portion 144 is connected to a cylindrical portion 152, the cylindrical portion having a proximal end 154. The proximal end 154 is housed in the second portion 142 of the valve structure 102 and is connected to the seal 138. The delivery conduit hub 106 will be discussed in more detail with reference to FIG. 3. The sealing surfaces described above rotate against themselves so as to not introduce unsterilized surfaces into contact with sterilized surfaces during rotation of the valve mechanism.

The cylindrical portion 152 may be connected to the second portion 142 of the valve structure 102 and housed within the second portion 142 such that the delivery conduit hub 106 is in fluid communication with the second portion 142. The proximal end 154 of the cylindrical portion 152 may be sealingly engaged with the seal 138 such that medicament passing through the seal 138 is directed through delivery conduit hub 106 and into the delivery conduit 150.

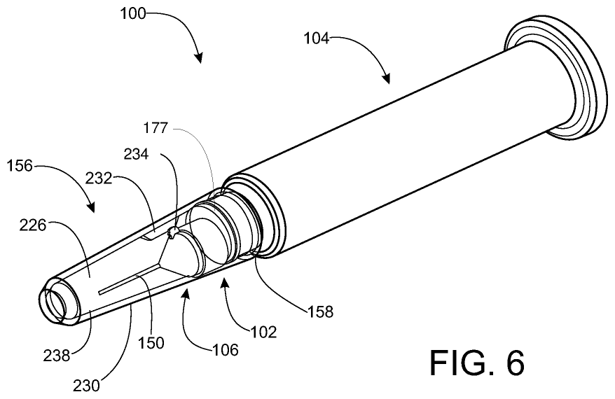

Optionally, the injection assembly 100 also includes a removable cap 156 that is configured to cover the delivery conduit 150, the delivery conduit hub 106, and the valve structure 102. The removable cap 156 has an open receiving end 158 to receive the delivery conduit 150, the delivery conduit hub 106, and the valve structure 102. The removable cap 156 may be closed at a distal end 160 opposite the receiving end 158. The removable cap 156 may maintain sterility of the delivery conduit 150, the delivery conduit hub 106, and the second portion 142 of the valve structure 102. The removable cap 156 may be made of a tough material, such as a thermoset or thermoplastic polymer, or any other suitable material including, but not limited to, elastomers. The removable cap 156 may protect the delivery conduit 150 from damage and/or reduce the likelihood of needle stick injury. The removable cap 156 may be transparent such that the delivery conduit 150 can be inspected without removing the cap 156. The removable cap 156 may comprise a detent 162 on its inner surface that provides positive engagement with the valve structure 136. The removable cap 156 will be discussed in more detail with reference to FIG. 6.

In FIG. 1, the valve structure 102 is shown as a component part of an injection assembly 100 including a medicament container 104, a delivery conduit hub 106, and a removable cap 156. The valve structure 102 may be provided pre-assembled as part of an injection assembly, or the valve structure 102 may be provided separately from the other components of the injection assembly such that it can be assembled into the injection assembly.

FIG. 2a shows an exploded perspective view of the valve structure 102 of FIG. 1, including the valve housing 136 and the seal 138. FIG. 2b shows a cross-section side-view of the valve structure of FIG. 2a. The first portion 140 of the valve housing 136 comprises a first generally cylindrical body 164 having a central longitudinal axis L4. The first generally cylindrical body 164 comprises an open end 168 and a connecting end 170. The first generally cylindrical body 164 has an outer surface 171. The outer surface 171 may comprise a Luer thread 172 (thread detail shown in FIG. 3) that corresponds to the thread 128 of the sleeve portion 126 such that the first generally cylindrical body 164 can be screwed into the male Luer fitting 124 to connect the valve structure 102 to the medicament container 104. The connecting end 170 is coupled to a flange 174. The flange 174 comprises a sleeve seat 176 that extends about the connecting end 170 of the first generally cylindrical body 164. When the valve structure 102 is connected to the medicament container 104, the sleeve seat 176 is sealingly engaged with the outer sealing surface 130 of the sleeve portion 126 of the medicament container 104. Optionally, the detent 162 on the removable cap 156 may snap-fit over the sleeve seat 176 to provide a positive engagement between the removable cap 156 and the valve structure 102. The sleeve seat 176 may be made of an elastomeric material such that the removable cap 156 may deform the sleeve seat 176 to create a sealing engagement which contains the delivery conduit hub 106 and delivery conduit 150. The sleeve seat 176 may be co-formed with the valve structure (for example by co-molding), or the sleeve seat 176 may comprise a sealing ring which is coupled to the outer sealing surface 130.

The second portion 142 comprises a second generally cylindrical body 178 having a central longitudinal axis L6. The second generally cylindrical body 178 comprises an open end 184, a connecting end 186, and an inner surface 182. The flange 174 is coupled to the connecting end 186 of the second generally cylindrical body 178. The first portion 140 and the second portion 142 are joined together such that the central longitudinal axis L4 of the first generally cylindrical body 164 is offset in a transverse direction from the central longitudinal axis L6 of the second generally cylindrical body 178. Alternatively, or additionally, the central longitudinal axis L4 of the first generally cylindrical body 164 may be non-coaxial with the central longitudinal axis L6 of the second generally cylindrical body 178. It will be understood that axes that are “offset in a transverse direction” will also be non-coaxial with one another. For example, two axes which are parallel but offset in a transverse direction will not intersect and will be non-coaxial. Two axes which intersect but which are not parallel will also be non-coaxial.

A first opening 192 extends from the first portion 140, through the flange 174 to the second portion 142. The first opening 192 has a central longitudinal axis L1 that is co-axial with the central longitudinal axis L4 of the first generally cylindrical body 164 and is offset in a transverse direction from the central longitudinal axis L6 of the second generally cylindrical body 178. The first opening 192 provides a fluid pathway between the first portion 140 and the second portion 142 such that the first portion 140 and the second portion 142 are in fluid communication with each other. The first opening 192 has a cylindrical wall 194 and may have a diameter equal to or greater than the outer diameter of the conduit portion 132 of the male Luer fitting 124. The stability of the connection between the valve housing 136 may be enhanced by providing the cylindrical wall 194 at a suitable diameter to slide over the conduit portion 132.

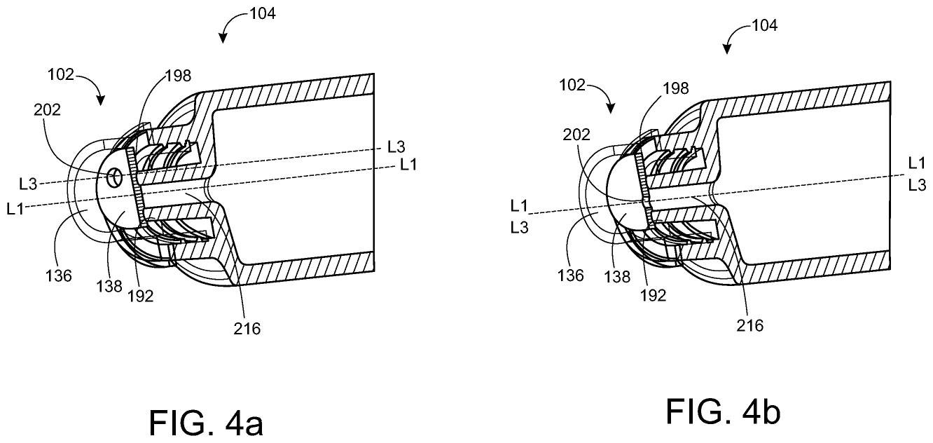

As shown in FIGS. 2a and 2b, the seal 138 is a circular disk comprising a first face 196, a second face 198, and a circumferential outer face 200. The seal 138 has a central longitudinal axis L2 running perpendicular to the first face 196 and the second face 198. When the seal 138 is seated in the second generally cylindrical body 178, the central longitudinal axis L2 of the seal 138 is co-axial with the central longitudinal axis L6 of the second generally cylindrical body 178 and is offset in a transverse direction from both the central longitudinal axis L4 of the first generally cylindrical body 164 and the central longitudinal axis L1 of the first opening 192. The seal 138 may be made of an elastomeric material. The seal 138 may be elastically deformable so as to be placed against a rigid component to create the sealing environment. The seal 138 further comprises a second opening 202 extending between the first face 196 and the second face 198. As shown in FIG. 2b, the second opening 202 is a through-hole with a cylindrical wall 204. The second opening 202 may have a central longitudinal axis L3 that is offset in a transverse direction from the central longitudinal axis L2 of the seal 138. The second opening 202 will be described in more detail with reference to FIG. 4a and FIG. 4b.

At a proximal end of the second portion 142, the first opening 192 is surrounded by a sealing seat 188. The sealing seat 188 comprises a sealing surface 190 that is flat and faces the open end 184 of the second generally cylindrical body 178. When the seal 138 is seated in the second generally cylindrical body 178, the second face 198 of the seal 138 may be in sealing engagement with the sealing surface 190 such that the seal 138 slidably seals the first opening 192. When the seal 138 is in sealing engagement with the sealing surface 190, the contact between the seal 138 and the sealing surface 190 prevents the passage of a fluid between the components under a reasonable pressure to be experienced in this application, for example, under the typical conditions, which may be applied to a syringe by pressure applied to a plunger of a medicament container. Slidably sealing means that whilst the surfaces are in sealing engagement with each other, the surfaces are arranged such that the components may rotate or slide relative to each other without breaking the sealing engagement. The seal 138 may have a diameter equal to or greater than the inner diameter of the second generally cylindrical body 178 such that an interference fit is formed when the seal 138 is seated in the second generally cylindrical body 178. The circumferential outer face 200 of the seal 138 may be in contact with the inner surface 182 of the second generally cylindrical body 178. The contact pressure between the inner surface 182 and the seal 138 from the interference fit may be sufficiently high so as to provide additional sealing.

The central longitudinal axis L1 of the first opening 192 may be a first central longitudinal axis L1. The central longitudinal axis L2 of the seal 138 may be a second central longitudinal axis L2. The central longitudinal axis L3 of the second opening 202 may be a third central longitudinal axis L3. The central longitudinal axis L4 of the first generally cylindrical body 164 may be a fourth central longitudinal axis L4.

As shown in FIG. 2b, the first central longitudinal axis L1, the second central longitudinal axis L2, and the third central longitudinal axis L3 are parallel to each other. The skilled person will understand that the first central longitudinal axis L1, the second central longitudinal axis L2, and the third central longitudinal axis L3 do not need to be exactly parallel to for the device to operate as intended, but may be substantially parallel so as to provide a suitable offset or non-coaxiality between the longitudinal axes.

FIG. 3 shows a cross-section perspective view of the injection assembly 100. Specifically, FIG. 3 shows how the valve structure 102, the medicament container 104, and the delivery conduit hub 106 with delivery conduit 150 are configured when they are assembled together. As shown in FIG. 3, the first portion 140 of the valve structure comprises a female Luer fitting. The first portion 140 is connected to the sleeve portion 126 of the male Luer fitting 124 such that the outer sealing surface 130 of the sleeve portion 126 is sealingly engaged with the sleeve seat 176. Whilst a Luer fitting has been shown in the figures, it will be appreciated that any suitable connection between the valve structure 102 and the medicament container 104 may be used including but not limited to a threaded fitting, a taper lock, a press fit, an O-ring, and/or adhesive or thermal bonding.

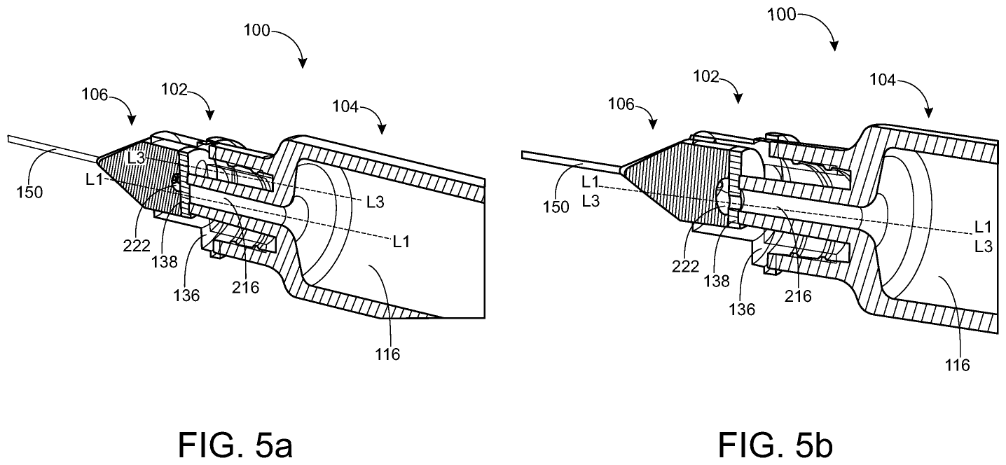

The medicament container comprises a third opening 216 that forms a channel through the conduit portion 132 of the Luer fitting 124. There is a fluid pathway between the interior volume 116 of the medicament container 104 and the third opening 216. The third opening 216 may be surrounded by the container sealing surface 134. The conduit portion 132 of the Luer fitting 124 extends from the distal end 110 of the medicament container 104, through the first generally cylindrical body 164 of the first portion 140, and into the first opening 192 of the valve structure 102. The third opening 216 provides a fluid pathway from the interior volume 116 of the medicament container 104 to the first opening 192 of the valve structure 102. The container sealing surface 134 may be in sealing engagement with the second face 198 of the seal 138 such that the seal 138 can seal off the fluid pathway between the medicament container 104 and the delivery conduit hub 104. A user may rotate the seal 138 into various positions that determine whether or not the seal 138 seals the fluid pathway between the medicament container 104 and the delivery conduit hub 106. The positions of the seal 138 will be described in more detail with reference to FIGS. 4a, 4b, 5a, and 5b.

As shown in FIG. 3, the delivery conduit hub 106 is housed within the second portion 142 of the valve structure 102. At its proximal end 154, the delivery conduit hub 106 comprises a connecting face 218 that is in fixed sealing engagement with the first face 196 of the seal 138. As such, rotation of the delivery conduit hub 106 will cause rotation of the seal 138 relative to the valve housing 136. The delivery conduit hub 106 may have a fifth central longitudinal axis L5 that is co-axial with the second central longitudinal axis L2 and offset from the first central longitudinal axis L1 and the third central longitudinal axis L3 in a transverse direction. The delivery conduit hub 106 and the seal 138 may be connected to each other in fixed sealing engagement using e.g., adhesives, co-molding, thermal bonding, or welding. The diameter of the cylindrical portion 152 of the delivery conduit hub 106 is to be less than or equal to the diameter of the inner surface 182 of the second generally cylindrical body 178 so that the delivery conduit hub 106 can be housed in the second portion 142. In FIG. 3, the cylindrical portion 152 is not in contact with the inner surface 182. However, the delivery conduit hub 106 may be sized such that the cylindrical portion 152 is in contact with the inner surface 182 as shown in FIG. 5a and to provide bearing support to the delivery conduit hub 106 from the inner surface 182.

The cylindrical portion 152 of the delivery conduit hub 106 comprises a chamber 222 that is in fluid communication with the second opening 202 (as shown in Figure The opening 148 of the delivery conduit hub 106 extends between the delivery conduit 150 and the chamber so as to provide fluid communication between the second opening 202 and the delivery conduit 150.

FIG. 4a is a cross-section perspective view of the medicament container 104 and the valve structure 102 where the seal 138 is in a first position. FIG. 4b shows the same view of FIG. 4a but where the seal 138 has been rotated into a second position. In the first position, the second opening 202 of the seal 138 is not aligned with the third opening 216 of the medicament container 104 and therefore does not allow fluid to be ejected from the medicament container 104. Therefore, in the first position, the seal 138 provides a barrier to fluid being ejected out of the medicament container 104 and seals the liquid medicament within the medicament container 104. In the second position, the second opening 202 of the seal 138 is aligned with the third opening 216 of the medicament container 104. Therefore, in the second position, the seal 138 no longer provides a barrier to fluid being ejected from the medicament container 104, and a liquid medicament may be ejected out of the medicament container 104 and through the second opening 202.

In FIG. 4a, the seal 138 is shown in the first position. When the seal 138 is in the first position, the second opening 202 is not in fluid communication with the first opening 192 of the valve structure 102 or with the third opening 216 of the medicament container 104. When the seal 138 is in the first position, the liquid medicament is sealed within the medicament container 104. The first position may be regarded as the ‘closed’ position or the ‘sealed’ position. As shown in FIG. 4a, the first position is the position at which the third central longitudinal axis L3 is offset from the first central longitudinal axis L1 as far as is possible by rotating the seal 138. However, it is not necessary that the first position be at such a ‘maximum offset’ position to achieve an adequate degree of sealing. The seal 138 may be rotated far enough such that the first central longitudinal axis L1 and the third central longitudinal axis L3 are sufficiently offset such that the second opening 202 does not overlap with either of the first opening 192 of the valve structure 102 or the third opening 216 of the medicament container 104.

FIG. 4b shows the seal 138 in the second position. When the seal 138 is in the second position, the second opening 202 of the seal 138 is in fluid communication with the first opening 192 of the valve structure 102. When the seal 138 is in the second position, the liquid medicament is not sealed within the medicament container 104 and can be ejected from the medicament container 104 through the second opening 202. The second position may be regarded as the ‘open’ position or ‘unsealed’ position. As shown in FIG. 4b, the second position is the position at which the third central longitudinal axis L3 is co-axial with the first central longitudinal axis L1. However, it is not necessary that the second position be such that the third central longitudinal axis L3 and the first longitudinal axis L1 are exactly aligned. The seal 138 may be rotated far enough such that the first central longitudinal axis L1 and the third central longitudinal axis L3 are sufficiently aligned such that the second opening 202 at least partially overlaps with either of the first opening 192 of the valve structure 102 or the third opening 216 of the medicament container 104.

The injection assembly 100 may be provided with the seal 138 in the first position in order to maintain sterility of the device and to seal the liquid medicament in the medicament container 104 the injection assembly 100 is to be used. A user may rotate the seal 138 from the first position to the second position in order to use the injection assembly 100. The operation of the injection assembly 100 will be described in more detail with reference to FIG. 6.

FIG. 5a shows a cross-section perspective view of the injection assembly 100 with the seal 138 in the first position (i.e., the closed, sealed position). There is a fluid pathway between the chamber 222 of the delivery conduit hub 106 and the delivery conduit 150 such that fluid which flows into the chamber 222 may pass to the delivery conduit 150. When the seal 138 is in the first position, the fluid pathway through the interior volume 116 and the third opening 216 is sealed off from the fluid pathway through the chamber 222 and the delivery conduit 150. Therefore, when the seal 138 is in the first position, there is no fluid communication between the interior volume 116 of the medicament container 104 and the delivery conduit 150.

FIG. 5b shows the same view as FIG. 5a, but where the seal 138 has been rotated into the second position (i.e., the open, unsealed position). When the seal 138 is in the second position, the fluid pathway through the interior volume 116 and the third opening 216 is in fluid connection with the fluid pathway through the chamber 222 and the delivery conduit 150. Therefore, there is a continuous fluid pathway and fluid communication between the interior volume 116 of the medicament container 104 and the delivery conduit 150.

As shown in FIG. 5a and FIG. 5b, the delivery conduit hub 106 and the seal 138 are fixedly connected such that they rotate together relative to the valve housing 136. In this way, a user may apply a rotation to the delivery conduit hub 106 thereby rotating the seal 138.

FIG. 6 shows a perspective view of the injection assembly 100 with the removable cap 156 in a covering position. The removable cap 156 is in the covering position when the removable cap 156 is covering the delivery conduit 150 and delivery conduit hub 106, and when the open end 158 of the removable cap 156 is sealed by the sleeve seat 176 or the sealing ring 177 of the valve structure 102. In the covering position, the removable cap 156 forms a sealed interior volume 226. The removable cap 156 may be rotatable relative to the valve housing 136 and the medicament container 104 such that rotation of the removable cap 156 effects rotation of the delivery conduit hub 106 and the seal 138.

The removable cap 156 may comprise an inner surface 228 and an outer surface 230. A first engagement member 232 may be disposed on the inner surface 228 wherein the first engagement member 232 is configured to engage a second engagement member 234 disposed on the delivery conduit hub 106. Since the first engagement member 232 may be engaged with the second engagement member 234, rotation of the removable cap may impart a torque on the delivery conduit hub such that the delivery conduit hub 106 rotates relative to the valve housing 136. The first engagement member 232 and the second engagement member 234 may each be formed as projections extending from the inner surface 228 and the delivery conduit hub 106, respectively. The first engagement member 232 and the second engagement member 234 each extend sufficiently far so as to contact each other when the removable cap 156 is in the covering position.

As shown in FIG. 6, the first engagement member 232 and the second engagement member 234 may be positioned such that the removable cap 156 can be rotated in a clockwise direction (from the perspective of a user looking at the tip of the delivery conduit 150), and thereby rotate the seal 138 from the first position to the second position. Once the seal 138 has been rotated to the second position, an anticlockwise rotation of the removable cap 156 shown in FIG. 6 will not impart a torque on the delivery conduit hub 106 unless the cap is rotated more than a full rotation. In this way, the removable cap 156 shown in FIG. 6 may allow a user to open the device but not to close it again. Optionally, the removable cap 156 and/or the delivery conduit hub 106 may include an additional engagement member (not shown) which would allow an anticlockwise rotation to impart a torque to rotate the seal 138 from the second position to the first position and therefore seal or close the device. Further optionally, the removable cap 156, the medicament container 104 and/or the valve structure 102 may include a mechanical stop configured to prevent rotation of the seal 138 and the delivery conduit hub 106 beyond the second position.

The valve structure 102 may be assembled by inserting the seal 138 into the second portion 142 of the valve housing 136 such that the seal 138 is in the first position. Optionally, the first portion 140 is fitted with the sealing ring 177. The delivery conduit hub 106 with the embedded delivery conduit 150 is fixed in sealing engagement with the seal 138. The delivery conduit hub 106 may be fixed in sealing engagement with the seal 138 either prior to or after inserting the seal 138 in the second portion 142 of the valve housing.

The valve structure 102 is connected to the medicament container 104 by mating the first portion 140, which is a female Luer fitting, with the male Luer fitting 124 fitting of the medicament container 104. It will be understood that the male Luer fitting and the female Luer fitting are interchangeable and may optionally be provided on the other of the medicament container 104 and the valve structure 102. Once the medicament container 104 is connected to the valve structure 102, the valve structure 102 is fixed (i.e., it does not substantially rotate) relative to the medicament container 104. Optionally, the removable cap 156 may be fitted over the delivery conduit hub 106 and the valve structure 102 to provide protection for the delivery conduit 150. Optionally, a plunger (not shown) may be inserted into the open back 118 of proximal end 112 of the medicament container 104.

The unique valve structure 102 allows sterility of the assembly to be maintained. The valve structure 102, the medicament container 104, and the delivery conduit hub 106 may all be sterilized together after assembly. Subsequently, the medicament container 104 may be filled with liquid medicament using a conventional filling process (filling from the proximal end 112). Since the valve structure 102 is provided with the seal 138 in the first position, the liquid medicament will be sealed from the fluid path 224 and kept in contact with only the medicament container 104 and the seal 138. The filled injection assembly 100 may be kept sterile until its usage. The injection assembly 100 may even maintain sterility after usage, depending on how the injection assembly 100 is used. For example, the valve may be closed after a first use and a new delivery conduit may be attached to the device, maintaining the sterility of the medicament container to be reused.

Alternatively, the valve structure 102 and the medicament container 104 can be connected to each other as a sub-assembly and then be sterilized independently from the delivery conduit hub 106. Prior to fully assembling the valve structure 102 and medicament container 104 sub-assembly with the delivery conduit hub 106, the medicament container 104 may be filled with liquid medicament using a conventional filling process. Alternatively, the medicament container 104 may be filled with liquid medicament via the valve structure 102 while maintaining sterility. To fill the medicament container 104 with liquid medicament via the valve structure 102, the seal 138 is first rotated to the second position, after which liquid medicament can be passed through the second opening 202 and into the medicament container 104. Once the liquid medicament has been passed through the second opening 202, the seal 138 can be rotated to the first position to seal the liquid medicament within the medicament container 104. Filling the medicament container 104 via the valve structure 102 allows a plunger to be inserted into the medicament container 104 prior to filling with liquid medicament. The valve structure 102 and medicament container 104 sub-assembly may maintain sterility for the entire shelf life of the medicament. The sterile valve structure 102 and medicament container 104 sub-assembly can be connected to the sterile delivery conduit hub 106 to form the injection assembly 100 without the need for further sterilization. This allows for different configurations of conduit deliveries or conduit delivery hubs to be optionally connected prior to use.

Alternatively, the valve structure 102 can be connected to the delivery conduit hub 106 as a sub-assembly and then sterilized independently from the medicament container 104. It is possible to pack the valve structure 102 and delivery conduit hub 106 sub-assembly in a conventional container nest so that the sub-assembly is sterile and ready for assembly with the sterile medicament container 104. After the injection assembly 100 is assembled, the medicament container 104 can be filled with liquid medicament using a conventional filling process.

Each of the assembly options detailed above provides a pre-filled and pre-loaded injection assembly 100 to the user. The pre-filled injection assembly 100 has sterile components (the valve structure 102, the medicament container 104, and the delivery conduit hub 106) and a sterile fluid path. An advantage of the described valve structure 102 is that the assembly 100 does not require septum piercing or any additional sterilisation steps by the user prior to use. The sterility of the components and the fluid path 224 can be maintained up to its usage or up to the end of the medicament's shelf life. The components and the fluid path 224 may even maintain their sterility after usage.

The operation of the injection assembly 100 will now be described with reference to FIG. 6. The injection assembly 100 can be supplied to the user fully assembled, filled with liquid medicament and fitted with a plunger. The injection assembly 100 may be provided to the user with the seal 138 in the first position. In the case that the injection assembly 100 is provided with the seal 138 in the first position, the first step in operation is to rotate the seal 138 from the first (closed) position to the second (open) position. In the case that the injection assembly 100 is supplied without the removable cap 156, the user first rotates the seal 138 by directly rotating the delivery conduit hub 106. The user may apply the torque through friction on the exposed portion of the delivery conduit hub 106. Alternatively, if the delivery conduit hub 106 is equipped with the second engagement member 234, then the user may leverage the second engagement member 234 to apply the necessary torque. Alternatively, the user may make use of a tool (not shown) that is configured to rotate the delivery conduit hub 106. The delivery conduit hub 106 is rotated either clockwise or anti-clockwise relative to the valve housing 136 until the seal 138 is in the second position.

In the case that the injection assembly 100 is supplied with the removable cap 156, the user can rotate the seal 138 by rotating the removable cap 156. The user may apply the torque through friction on the outer surface 230 of the removable cap 156. By rotating the removable cap 156 such that the first engagement member 232 engages the second engagement member 234, the necessary torque can be provided by the user to rotate the delivery conduit hub 106. The removable cap 156 is rotated either clockwise or anti-clockwise until the seal 138 is in the second position. The user then removes the cap 156 by pulling the removable cap 156 away from the medicament container 104 and towards the distal direction. Alternatively, the removable cap 156 and the delivery conduit hub 106 may be configured so that simply pulling the removable cap 156 away from the medicament container will rotate the seal 138 from the first position to the second position. In such an embodiment, the first engagement member 232 and the second engagement member 234 may extend around the inner surface of the removable cap 156 in a transverse direction so as to provide a cam which imparts a torque to the delivery conduit hub 106 and the seal 138.

After the seal 138 is rotated from the first position to the second position, and after the removable cap 156 has been removed (in the optional case that the removable cap 156 is included), the injection assembly 100 is ready to be used to administer the liquid medicament. The distal end 151 of the delivery conduit 150 may be inserted at an injection site (e.g., a body or an intravenous bag). A plunger is pushed from the proximal end 112 of the medicament container 104 to the distal end 110 to eject the liquid medicament from the interior volume 116 to the delivery conduit 150 and to the injection site. The user may choose to dispense either all of the liquid medicament or only a portion of the liquid medicament. In the case that only a portion of the liquid medicament is dispensed, the seal 138 can be rotated from the second position back to the first position by rotating the seal 138 from the second position to the first position. Rotating the seal 138 from the second position to the first position after dispensing a portion of the liquid medicament seals the remaining liquid medicament in the medicament container 104 and allows conservation of the remaining liquid medicament for further use. If sterility requirements permit, the operation steps of the injection device 100 above can be repeated to dispense any remaining liquid medicament.

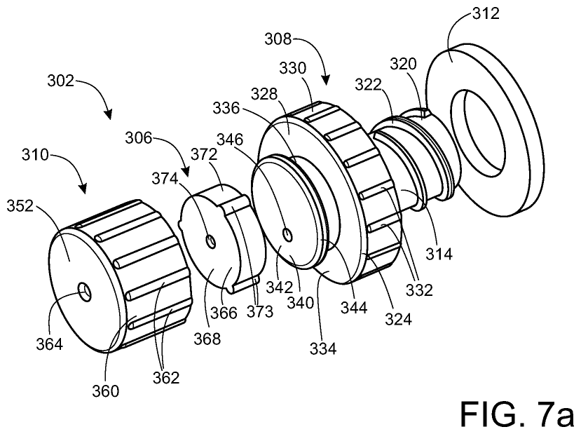

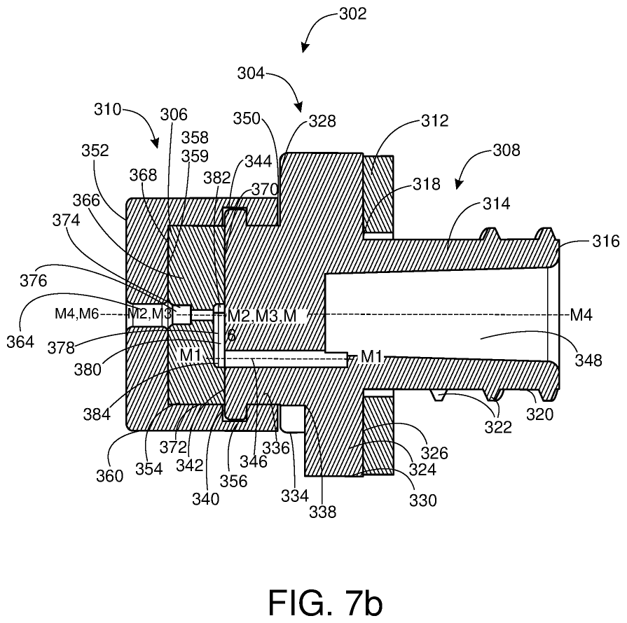

FIG. 7a shows an exploded perspective view of an embodiment of a valve structure 302 according to a further embodiment. FIG. 7b shows a cross-section side-view of the valve structure 302 of FIG. 7a. The valve structure 302 comprises a valve housing 304, a seal 306, and an optional sealing ring 312. The valve structure 302 is configured to connect to a medicament container and a delivery conduit hub or a delivery conduit (not shown). The valve structure 302 may be compatible with the medicament container 104 and the delivery conduit hub 106 of the embodiment illustrated in FIG. 1 and may be assembled into an injection assembly 100.

The valve housing 304 comprises a first portion 308 and a second portion 310. The first portion 308 comprises a first sub-portion 314 that is configured to connect to the distal end 110 of a medicament container 104 via a male Luer fitting 124 on the medicament container 104. The first sub-portion 314 comprises a cylindrical body having an open end 316, a connecting end 318, and an outer surface 320. The outer surface 320 may comprise a continuous Luer thread 322 that corresponds to the thread 128 of the sleeve portion 126 of the male Luer fitting 124 such that the first sub-portion 314 can be screwed into the male Luer fitting 124.

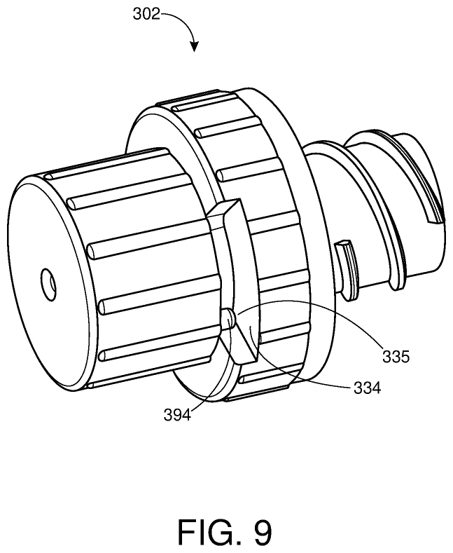

The first portion 308 further comprises a second sub-portion 324. The second sub-portion comprises a cylindrical body having an outer diameter greater than an outer diameter of the first sub-portion 314. The second sub-portion 324 comprises a proximal side 326 and a distal side 328. The connecting end 318 of the first sub-portion 314 is rigidly connected to the proximal side 326 of the second sub-portion 324. The second sub-portion 324 has an outer surface 330 which may comprise protruding ribs 332 that to provide grip for a user during operation of the device. Optionally, the distal side 328 may comprise a slot 334 that is configured to provide a stop assembly 335. The slot 334 and stop assembly 335 will be described in more detail with reference to FIG. 9.

The first portion 308 further comprises a third sub-portion 336 connected to the second portion 310. The third sub-portion 336 comprises a cylindrical body having an outer diameter that is less than the outer diameter of the second sub-portion 324. The third sub-portion 336 has a proximal side 338 fixedly connected to the distal side 328 of the second sub-portion 324. The third sub-portion 336 has a distal side 340 that comprises a flat sealing surface 342. The sealing surface 342 is surrounded by a flange 344 that rotatably couples the first portion 308 to the second portion 310. The first sub-portion 314, the second sub-portion 324 and the third sub-portion 336 all share a central longitudinal axis M4.

The first portion 308 further comprises a first opening 346 having a central longitudinal axis M1 that is offset in a transverse direction to the central longitudinal axis M4 of the first sub-portion. The first opening 346 extends from the sealing surface 342 of the third sub-portion 336 and through the second sub-portion 324. The first portion 308 comprises a Luer opening 348 that extends from the open end 316 of the first sub-portion 314 through to at least part of the second sub-portion 324. The Luer opening has a central longitudinal axis that is co-axial with the central longitudinal axis M4 of the first sub-portion. The Luer opening 348 is configured to receive the conduit portion 132 of the male Luer fitting 124. The first opening 346 is in fluid communication with the Luer opening 348 such that there is a fluid path provided between the open end 316 of the first sub-portion 314 and the sealing surface 342 of the third sub-portion 336.

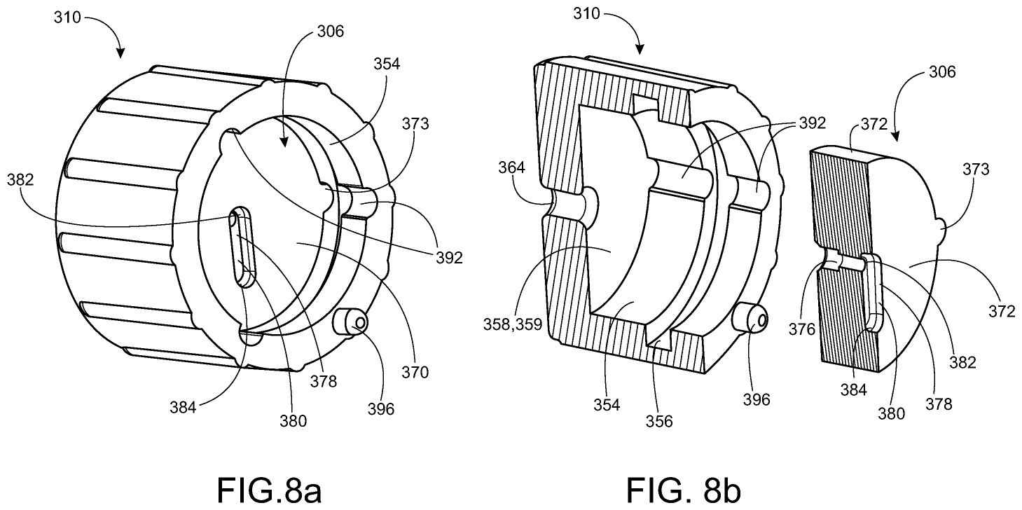

The second portion 310 comprises a hollow cylindrical body having a central longitudinal axis M6. The second portion 310 comprises an open end 350 and a closed end 352. The second portion has an inner cylindrical wall 354 that comprises a retaining slot 356 configured to receive the flange 344 of the third sub-portion 336. The second portion 310 may be made of a flexible material such that the retaining slot 356 can be snap-fit to the flange 344. The interior of the closed end 352 includes a flat sealing seat 358 against which the seal 306 may be seated. The sealing seat 354 provides a sealing surface 359. The connection of the seal 306 to the second portion 310 will be described in more detail with reference to FIG. 8a and FIG. 8b. The second portion 310 comprises an outer cylindrical surface 360 including protruding ribs 362 for a user to grip during operation of the device.

The second portion 310 comprises a delivery opening 364 that extends between the sealing seat 358 and the outer surface of the closed end 352. The delivery opening 364 has a central longitudinal axis that is co-axial with the central longitudinal axis M6 of the second portion 310. The delivery opening 364 may be tapered such that a delivery conduit may be retained within the delivery opening 364. The delivery conduit (not shown) may optionally take the form of a needle or a flexible tube.

The seal 306 comprises a cylindrical body 366 having a central longitudinal axis M2 that is co-axial with the central longitudinal axis M6 of the second portion 310. The seal 306 comprises a first face 368, a second face 370, and a circumferential outer face 372. At least one key 373 may be disposed on the circumferential outer face 372.As shown in FIG. 7a, three keys 373 are disposed on the circumferential outer face 372. The keys 373 extend in a direction perpendicular to the first face 368 and the second face 370. The key 373 will be described in more detail with reference to FIG. 8a and FIG. 8b. The seal further comprises a second opening 374. The second opening 374 comprises a central channel 376 having a central longitudinal axis M3, and a connecting channel 378 that is in fluid communication with the central channel 376. The central channel 376 extends from the first face 368 and at least partially through the body 366 of the seal 306. The connecting channel 378 comprises a slot 380 extending from the second face 370 to the central channel 376. Specifically, the slot 380 runs radially along the second face 370 from a first end 382 that coincides with the central longitudinal axis M2 of the seal 306 to a second end 384 that is radially offset from the central longitudinal axis M2 of the seal 306.

The seal 306 is housed within the second portion 310. Specifically, the first face 368 of the seal 306 is in sealing engagement with the sealing seat 358 of the second portion 310. In this position, the central channel 376 of the seal 306 is in fluid communication with the delivery opening 364. The diameter of the circumferential outer face 372 of the seal 306 is equal to or less than a diameter of the inner cylindrical wall 354. When the second portion 310 is rotatably coupled to the first portion 308 via the flange 344 and the retaining slot 356, the second face 370 of the seal 306 is held in sealing engagement with the sealing surface 342 of the third sub-portion 336. Since the flange 344 is free to slide within the retaining slot 356, the user may rotate the seal 306 to open or close the fluid pathway between the Luer opening 348 and the delivery opening 364.

An optional sealing ring 312 formed of an elastomeric material may be provided. The sealing ring 312 surrounds the first sub-portion 314 and sits against the proximal side 326 of the second sub-portion 324 such that the container sealing surface 134 of the sleeve portion 126 of the male Luer fitting 124 can be sealed against the sealing ring 312 when the valve structure 102 is connected to the medicament container 104.

The central longitudinal axis M1 of the first opening 346 may be a first central longitudinal axis M1. The central longitudinal axis M2 of seal 306 may be a second central longitudinal axis M2. The central longitudinal axis M3 of the central channel 376 of the second opening 374 may be a third central longitudinal axis M3. The central longitudinal axis M4 of the first sub-portion 314 may be a fourth central longitudinal axis M4.

As shown in FIG. 7b, the first central longitudinal axis M1, the second central longitudinal axis M2, and the third central longitudinal axis M3 are parallel to each other. The skilled person will understand that the first central longitudinal axis M1, the second central longitudinal axis M2, and the third central longitudinal axis M3 do not have to be exactly parallel for the device to function properly. The fourth central longitudinal axis M4 is co-axial with the second central longitudinal axis M2 and the third central longitudinal axis M3 and is offset from the first central longitudinal axis M1 in a transverse direction. In the illustrated embodiment, when the valve structure 302 is connected to the medicament container 104, the valve structure 302 can be considered to have a central longitudinal axis that is generally co-axial with a central longitudinal axis of the medicament container 104 and therefore the delivery conduit or needle will be centrally located with respect to the medicament container.

FIG. 8a shows a perspective view of the seal 306 housed within the second portion 310 of the valve housing 304 as viewed from the open end 350. FIG. 8b shows an exploded cross-section perspective view of the seal 306 and the second portion 310 of FIG. 8a. As shown, the inner cylindrical wall 354 of the second portion 310 comprises at least one keyway 392 running parallel to the central longitudinal axis M6 of the second portion 310 from the open end 350 to the sealing seat 358. The at least one keyway 392 corresponds to the at least one key 373 provided on the seal 306. The at least one keyway 392 extends across the entire inner cylindrical wall 354 such that the seal 306 may be inserted into the second portion 310 from the open end 350 and displaced until the first face 368 is in sealing engagement with the sealing seat 358. The at least one keyway 392 and corresponding key 373 provide for the seal 306 to be fixedly coupled to the second portion 310, and the seal 306 is therefore rotatable together with the second portion 310 relative to the first portion 308.

In the illustrated embodiment, when the seal 306 is in the first position, the second opening 364 is not in fluid communication with the first opening 346. When the seal 306 is in the first position, the liquid medicament is sealed within the medicament container 104. The first position can also be referred to as the ‘closed’ position or the ‘sealed’ position. In combination, the first opening 346, the central channel 376, and the connecting channel 378 are arranged such that when the seal 306 is rotated to a position relative to the valve housing 304 where there is no overlap between the connecting channel 378 and the first opening 346, there is no fluid communication between the second opening 374 and the first opening 346. When the valve structure 302 is connected to the medicament container 104 and there is no fluid communication between the second opening 374 and the first opening 346, the liquid medicament is sealed within the medicament container 104.

When the seal 306 is in the second position, the second opening 364 is in fluid communication with the first opening 346. When the seal 306 is in the second position, the liquid medicament is not sealed within the medicament container 104 and can be ejected from the medicament container 104 through the second opening 374 and the delivery opening 364. The second position may be referred to as the ‘open’ position or ‘unsealed’ position. The second position of the seal 306 may be defined by the seal 306 being rotated to a position where the second end 384 of the slot 380 coincides with the first central longitudinal axis M1 such that there is an overlap between the slot 380 and the first opening 346.

The seal 306 may be rotated between the first position and the second position by the user. The injection assembly 100 may be provided with the seal 306 in the first position so that the liquid medicament can remain sealed within the medicament container 104 until a user uses the injection assembly 100. The user may rotate the seal 306 from the first position to the second position in order to use the injection assembly 100. Because the seal 306 is fixedly coupled to the second portion 310, the user may control the rotation of the seal 306 by controlling the rotation of the second portion 310.

Optionally, a stop assembly 335 may be provided. As shown in FIG. 9, the second portion 310 may comprise a pin 394, and the first portion 308 may comprises a corresponding slot 334. The slot 334 and the pin 394 may be configured such that the slot 334 prevents the movement of the pin 394 beyond a limit when the second portion 310 is rotated. The stop assembly 335 may limit the rotation of the second portion beyond the first position or the second position. While FIGS. 7a to 9 illustrate the stop assembly 335 wherein the first portion 308 comprises a slot 334 and the second portion 310 comprises a pin 394, alternatively, the second portion 310 may comprise the slot 334 and the first portion 308 may comprise the corresponding pin 394.

The assembly process of the injection assembly 100 will now be described. The valve structure 302 is assembled by first inserting the seal 306 into the second portion 310 of the valve housing 304 such that the first face 368 of the seal 306 is in sealing engagement with the sealing surface 359 of the sealing seat 358 and is keyed in the at least one keyway 392. The second portion 310 is then connected to the first portion 308 by snap-fitting the retaining slot 356 of the second portion 310 to the flange 344 of the first portion 308 such that the second face 370 of the seal 306 is in sealing engagement with the sealing surface 342 of the third sub-portion 336. Optionally, the first portion 308 is fitted with the sealing ring 312.

The valve structure 302 is connected to the medicament container 104 by mating the first portion 308, which is a female Luer fitting, with the male Luer fitting 124 fitting of the medicament container 104 such that the conduit portion 132 is housed within the Luer opening 348. It will be understood that the male Luer fitting and the female Luer fitting are interchangeable and may optionally be provided on the other of the medicament container 104 and the valve structure 302. Once the medicament container 104 is connected to the valve structure 302, the valve structure 302 is fixed (i.e., it does not substantially rotate) relative to the medicament container 104. Optionally, a plunger (not shown) may be inserted into the open back 118 of proximal end 112 of the medicament container 104.

A delivery conduit may be connected to the delivery opening 364.

The valve structure 302, the medicament container 104, and the delivery conduit hub 106 may all be sterilized independently before assembly or together after assembly. The medicament container 104 can be filled with liquid medicament using a conventional filling process, i.e., filling via the proximal end 112. Alternatively, the medicament container 104 can be filled with liquid medicament via the delivery opening 364 in the valve structure 302 with the seal 306 in the second position.

The injection assembly 100 can be supplied fully assembled, filled with liquid medicament, and fitted with a plunger. The injection assembly 100 may be provided with the seal 306 in the first position. In the case that the injection assembly 100 is provided with the seal 306 in the first position, the first step in operation is to rotate the seal 306 from the first (closed) position to the second (open) position. In the case that the injection assembly 100 is supplied with a removable cap 156, the removable cap 156 is first removed by pulling the cap 156 away from the medicament container 104. The user may rotate the seal 138 by directly rotating the second portion 310. The user may apply a torque through friction on the outer cylindrical surface 360 and the protruding ribs 362 of the second portion 310.

After the seal 306 is rotated from the first position to the second position, the injection assembly 100 is ready to be used to administer the liquid medicament to the patient. The distal end 151 of the delivery conduit 150 is inserted into an injection site (e.g., a body that requires the liquid medicament).

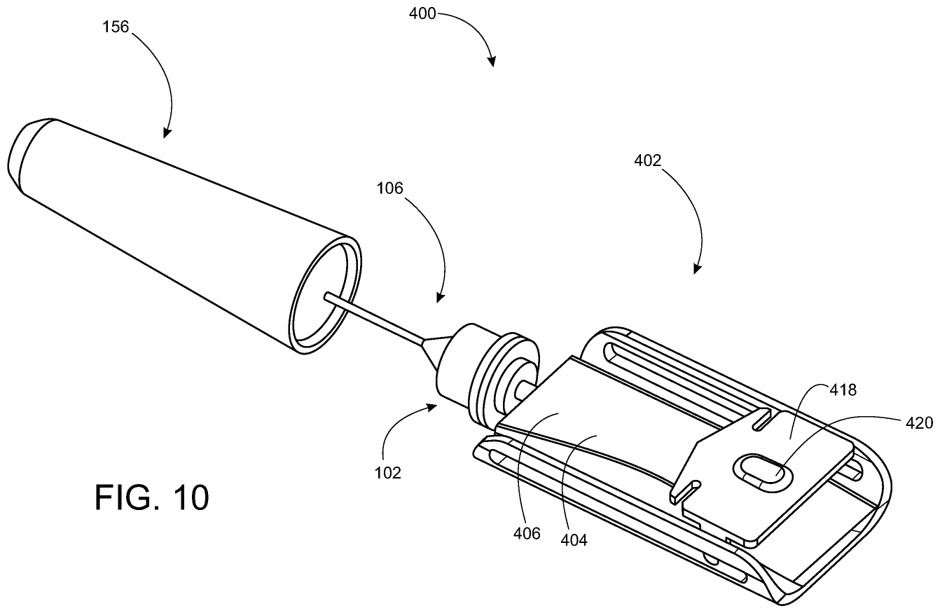

FIG. 10 shows a perspective view of an embodiment of the injection assembly 400 comprising a flexible medicament container 402. The flexible medicament container 402 may be compatible with the valve structure 102 and the delivery conduit hub 106 of the injection assembly 100. Additionally, or alternatively, the flexible delivery conduit 502 may be compatible with the valve structure 302 of the injection assembly 100.

The flexible medicament container 402 comprises a flexible bladder 404 including a skin 406 containing liquid medicament. The skin 406 may be made of a flexible material such that the bladder 404 may be compressed. The flexible bladder 404 also comprises a neck 407 configured to connect to a valve structure such as the valve structure 102.

The flexible medicament container 402 comprises a push tab 418 that can be used to squeeze the liquid medicament out of the flexible bladder 404. The push tab 418 has a ridge 420 to provide leverage to a user during operation.

In the case that the flexible medicament container 402 is fitted with the valve structure 102, the flexible medicament container 402 may be filled with liquid medicament via the valve structure 102.

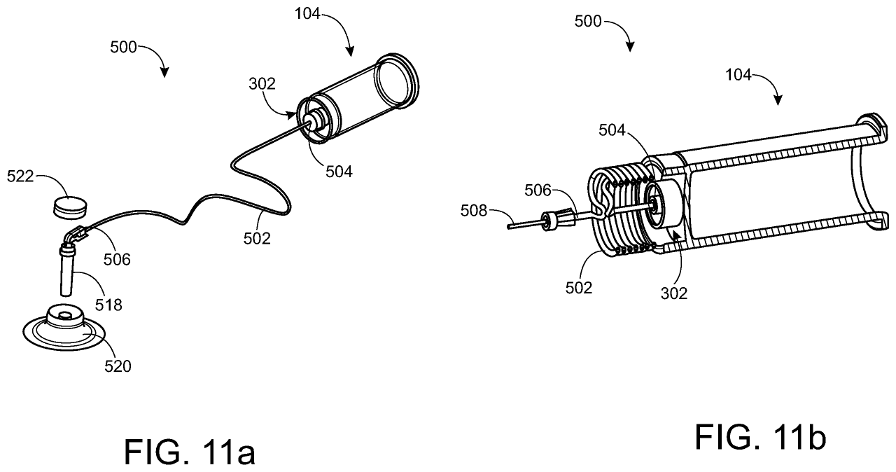

FIG. 11a and FIG. 11b show a perspective view of an injection assembly 500 according to a further embodiment. Injection assembly 500 comprises a flexible delivery conduit 502. FIG. 11a shows the flexible delivery conduit 502 in a deployed configuration, and FIG. 11b shows the flexible delivery conduit in a stored configuration. The flexible delivery conduit 502 may be compatible with the medicament container 104, the valve structure 102, and the delivery conduit hub 106 of injection assembly 100. Additionally, or alternatively, the flexible delivery conduit 502 may be compatible with the valve structure 302 of the injection assembly 100 and the flexible medicament container 402 of injection assembly 400.

The flexible delivery conduit 502 has a proximal end 504 and a distal end 506. The proximal end 504 is configured to connect to a delivery conduit hub 106, and the distal end 506 is configured to connect to a remote injection needle 508. Alternatively, as shown in the FIG. 11a and FIG. 11b, the proximal end 504 may be configured to connect to a valve structure 302. The needle 508 and the collar 514 may be covered by a cap 518. The needle 508 and the collar 514 may be configured to connect to an infusion set body 520. The infusion set body 520 may comprise an infusion set cap 522 that is configured to cover the infusion set body 520 prior to connecting with the needle 508 and the collar 514.

The injection assembly 500 may be provided to the user with the flexible delivery conduit 502 in the coiled position as shown in FIG. 11b. The user may uncoil the flexible delivery conduit 502 and connect the needle 508 to the infusion set body 520.

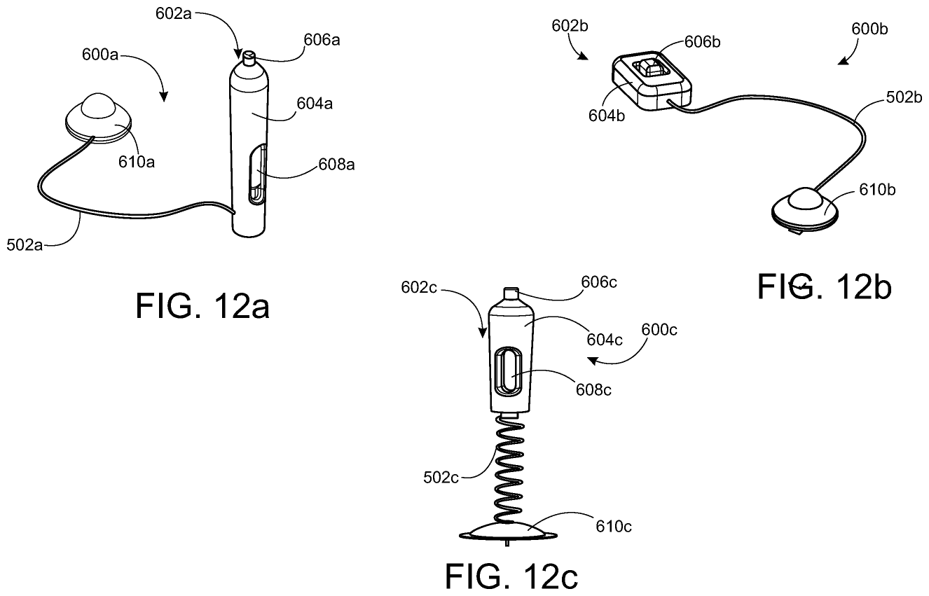

FIG. 12a, FIG. 12b and FIG. 12c show perspective views of alternative injection assemblies 600a, 600b, 600c, respectively, according to further embodiments. As shown, each of the medicament containers 602a-602c respectively comprises an injector body 604a-604c, with an activation button 606a-606c. In FIG. 12a and FIG. 12c, each of the injector bodies 604a and 604c are shaped as a barrel to be held in the hand and each having a viewing window 608a and 608c, respectively. The viewing windows 608a and 608c allow the user to view the state of the liquid medicament, for example, the volume or the colour of the liquid medicament. In FIG. 12b, the injector body 604b is box-shaped and configured to be positioned on a surface. The injection assemblies 600a, 600b, 600c each comprise a flexible delivery conduit 502a, 502b, 502c, respectively, and may be connected to a respective infusion set body 610a, 610b, 610c.

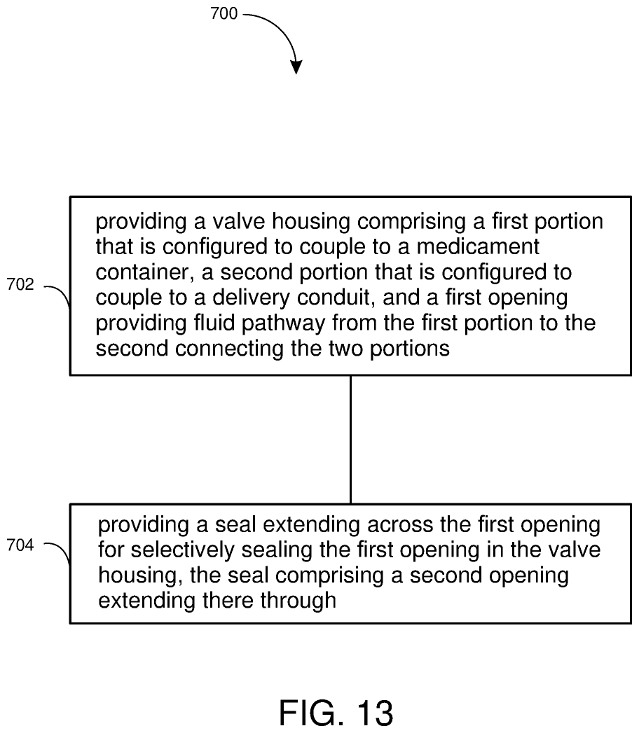

FIG. 13 is a flowchart illustrating a method 700 for manufacturing a valve structure for selectively connecting the medicament container to the delivery conduit. The valve structure may be the valve structure 102 of injection assembly 100 or the valve structure 302 of injection assembly 100.

A first step 702 comprises providing a valve housing comprising a first portion that is configured to couple to a medicament container, a second portion that is configured to couple to a delivery conduit, and a first opening providing fluid pathway from the first portion to the second portion.

A second step 704 comprises providing a seal extending across the first opening for selectively sealing the first opening in the valve housing, the seal comprising a second opening extending therethrough.

In some further aspects, the valve structures described throughout this application may be used to introduce one or more medicament components into the medicament container via the described connection engagement.

The preceding detailed description describes structures and methods for a rotatable valve structure that can be moved between an open position and closed position in an injection assembly. However, the skilled person will understand that the invention is not limited to the exemplary device described here. Rather, one or more benefits associated with the present invention may be implemented in connection with other drug delivery systems, such as a wearable infusion device, autoinjectors, or an IV infusion set, as will be apparent to the skilled person in light of the preceding detailed description.

It will also be understood that, where used, the terms “proximal”, “distal”, “side”, “end”, “open” and “closed” are used for convenience in interpreting the drawings and are not to be construed as limiting. The term “comprising” should be interpreted as meaning “including but not limited to”, such that it does not exclude the presence of features not listed.

The embodiments shown in the figures show Luer lock fittings, but the skilled person would understand that a Luer slip may be used in place of a Luer lock fitting.

The embodiments described and shown in the accompanying drawings above are provided as examples of ways in which the invention may be put into effect and are not intended to be limiting on the scope of the invention. Modifications may be made, and elements may be replaced with functionally and structurally equivalent parts, and features of different embodiments may be combined without departing from the disclosure.

What is claimed:

1. A valve structure for selectively connecting a medicament container to a delivery conduit, the valve structure comprising:
a valve housing comprising a first portion that is configured to couple to a medicament container, a second portion that is configured to couple to a delivery conduit, and a first opening providing a fluid pathway from the first portion to the second portion; and
a seal extending across the first opening for selectively sealing the first opening in the valve housing, the seal comprising a second opening extending therethrough,
wherein the seal is rotatable relative to at least one of the first portion and the second portion of the valve housing between a first position in which the second opening is not in fluid communication with the first opening and a second position in which the seal is rotated such that the second opening is in fluid communication with the first opening.

2. The valve structure of claim 1, wherein the at least one of the first portion and the second portion of the valve housing further comprises a sealing seat surrounding the first opening in the valve housing, against which the seal is seated.

3. The valve structure of claim 1, wherein the second portion is coupled to a delivery conduit via a delivery conduit hub, and wherein the second opening in the seal is in fluid communication with the delivery conduit.

4. The valve structure of claim 1, wherein:
the first opening has a first central longitudinal axis,
the seal has a second central longitudinal axis,
the second opening has a third central longitudinal axis, and
wherein the second central longitudinal axis is offset from the first and third central longitudinal axes in a transverse direction, or non-coaxial with the first and third central longitudinal axes.

5. The valve structure of claim 1, wherein:
the seal is fixedly coupled to the second portion of the valve housing, and wherein the seal and the second portion of the valve housing are rotatable together relative to the first portion of the valve housing.

6. The valve structure of claim 5, wherein the seal and the second portion of the valve housing are keyed such that they are rotatable together.

7. The valve structure of claim 1, wherein:
the second opening comprises a central channel and a connecting channel that is in fluid communication with the central channel.

8. The valve structure of claim 7, wherein the second opening is arranged such that when the seal is in the second position, the connecting channel is in fluid communication with the first opening and there is fluid communication between the first opening and the central channel, and
when the seal is in the first position, the connecting channel is not in fluid communication with the first opening and there is no fluid communication between the first opening and the central channel.

9. The valve structure of claim 7, wherein the seal comprises a body having a first face, and wherein the central channel extends from the first face and at least partially through the body of the seal.

10. The valve structure of claim 9, wherein the body of the seal has a second face, and wherein the connecting channel comprises a slot extending from the second face to the central channel.

11. The valve structure of claim 10, wherein the slot extends radially from a central axis of the seal to an eccentric position.

12. The valve structure of claim 1, wherein the first portion comprises a flange, the second portion comprises a corresponding detent, and wherein the flange and the detent are configured to rotatably couple the first portion and the second portion.

13. The valve structure of claim 1, further comprising a stop assembly configured to limit rotation of the seal between the first position and the second position.

14. The valve structure of claim 13, wherein the stop assembly comprises a pin fixed to one of the first portion and the second portion, the pin being received in a corresponding slot of the other of the first portion and the second portion, such that the slot restricts rotation of the second portion beyond the first position or the second position.

15. The valve structure of claim 4, wherein the first central longitudinal axis, the second central longitudinal axis and the third central longitudinal axis are parallel to each other.

16. The valve structure of claim 3, wherein the delivery conduit hub is a needle hub comprising a needle.

17. The valve structure of claim 16, wherein the needle is aligned with the second central longitudinal axis of the seal.

18. The valve structure of claim 1, wherein the seal is a circular disc.

19. The valve structure of claim 1, wherein the first portion comprises a first generally cylindrical body and/or wherein the second portion comprises a second generally cylindrical body.

20. The valve structure of claim 19, wherein the first generally cylindrical body comprises a fourth central longitudinal axis and is substantially co-axial with the first central longitudinal axis.

21. The valve structure of claim 2, wherein the sealing seat provides a sealing surface disposed in the second portion of the valve housing.

22. The valve structure of claim 21, wherein the second opening is in registry with the sealing surface when the seal is in the first position.

23. The valve structure of claim 1, wherein the first portion comprises a connector for coupling the valve structure to a medicament container.

24. The valve structure of claim 1, wherein the seal comprises an elastomeric material.

25. A device for delivering medicament comprising:
a valve structure having:
a valve housing comprising a first portion that is configured to couple to a medicament container, a second portion that is configured to couple to a delivery conduit, and a first opening providing a fluid pathway from the first portion to the second portion; and
a seal extending across the first opening for selectively sealing the first opening in the valve housing, the seal comprising a second opening extending therethrough,
wherein the seal is rotatable relative to at least one of the first portion and the second portion of the valve housing between a first position in which the second opening is not in fluid communication with the first opening and a second position in which the seal is rotated such that the second opening is in fluid communication with the first opening; and
a medicament container comprising a third opening coupled to the first portion and in fluid communication with the first opening in the valve housing.

26. The device of claim 25, wherein the third opening is surrounded by a container sealing surface and wherein the seal is configured to seal against the container sealing surface.

27. The device of claim 25, wherein one of the medicament container and the second portion of the valve housing comprises a male luer connecting portion and wherein the other of the medicament container and the second portion of the valve housing comprises a female luer connecting portion.

28. The device of any of claim 25, further comprising an injection needle in fluid communication with the delivery conduit hub.

29. The device of claim 28, wherein the injection needle is remote from the delivery conduit hub and is connected thereto via a flexible delivery conduit.

30. The device of claim 25, further comprising a removable cap.

31. The device of claim 30, wherein the removable cap is configured to rotate the seal from the first position to the second position.

32. The device of claim 31, wherein the cap is removable by rotation and is configured to rotate the seal between the first and second position.

33. The device of claim 30, wherein:
the removable cap comprises a first engagement member;
the valve structure comprises a second engagement member,
wherein the first engagement member is configured to engage the second engagement member during removal of the cap to move the seal from the first position to the second position.
